(19) United States
(12) Patent Application Publication
(10) Pub. No.: US 2013/0208863 A1
(43) Pub. Date: Aug. 15, 2013

(54) MEDICAL DIAGNOSTIC IMAGING APPARATUS

(71) Applicants: Kabushiki Kaisha Toshiba, (US); Toshiba Medical Systems Corporation, (US)

(72) Inventors: Yuichiro WATANABE, Yaita-shi (JP); Masanori MATSUMOTO, Nasushiobara-shi (JP); Ryuji ZAIKI, Utsunomiya-shi (JP); Reiko HASHIMOTO, Yaita-shi (JP); Kazuo IMAGAWA, Nasushiobara-shi (JP)

(73) Assignees: TOSHIBA MEDICAL SYSTEMS CORPORATION, Tochigi-ken (JP); KABUSHIKI KAISHA TOSHIBA, Minato-ku (JP)

(21) Appl. No.: 13/842,130

(22) Filed: Mar. 15, 2013

Related U.S. Application Data
(63) Continuation of application No. PCT/JP2012/058580, filed on Mar. 30, 2012.

(30) Foreign Application Priority Data
Mar. 30, 2011 (JP) 2011-073799

(51) Int. Cl. A61B 6/12

(52) U.S. Cl. CPC A61B 6/12
USPC 378/62

(57) ABSTRACT

A medical diagnostic imaging apparatus of an embodiment includes: an imaging unit configured to capture a medical image of a blood vessel in which an artificial valve is to be placed; a display unit configured to display the medical image captured by the imaging unit; a storage unit configured to store artificial valve information about a length of the artificial valve; and a control unit configured to find a length of the artificial valve in the medical image displayed by the display unit, and to judge whether or not the length thus found and the length in the artificial valve information stored in the storage unit are the same.

### CROSS-REFERENCE TO THE RELATED APPLICATION

This application is based on and claims the benefit of priority from International Application No. PCT/JP2012/058580, filed on Mar. 30, 2012 and Japanese Patent Application No. 2011-073799, filed on Mar. 30, 2011; the entire contents of which are incorporated herein by reference.

### FIELD

Embodiments described herein relate generally to a medical diagnostic imaging apparatus and relate, for example, to a medical diagnostic imaging apparatus configured to display a medical image of a part of interest of a subject.

### BACKGROUND

Medical diagnostic imaging apparatuses include an imaging unit configured to image a subject on a top panel of a bed, a C arm configured to hold the imaging unit, and the like, and are configured to move the imaging unit to an imaging position for the subject on the top panel, capture a medical image, i.e., a radiograph, of a given part of the subject with the imaging unit, and display the radiograph on a monitor.

These medical diagnostic imaging apparatuses have been used in valve replacement for placing an artificial valve inside a subject's blood vessel. This valve replacement is the replacement of an impaired valve (e.g., mitral valve, aortic valve, etc.) with an artificial valve such as a carbon mechanical valve or an animal's valve. Conventional valve replacement treatment has been performed through surgical methods. In recent years, however, percutaneous treatment using a catheter has been established and drawing attention for its less invasive nature. In valve replacement, importance is place on not only the position to place an artificial valve but also the parallelism between the blood vessel and the artificial valve. Thus, the aforementioned radiograph is used to check the position and the parallelism.

However, because checking the position of the artificial valve through the radiograph, the surgeon can check the parallelism between the blood vessel and the artificial valve only in a plan view on a monitor. Thus, it is difficult to check the parallelism in the depth direction of the blood vessel. If one uses the aforementioned medical diagnostic imaging apparatus to check the parallelism between the blood vessel and the artificial valve in the depth direction of the blood vessel, imaging needs to be performed multiple times by changing the imaging position along the circumference of the blood vessel. This requires time and effort.

### BRIEF DESCRIPTION OF THE DRAWINGS

FIG. 1 is a diagram showing a schematic configuration of a medical diagnostic imaging apparatus according to an embodiment.

FIG. 2 is a flowchart showing the flow of a process of assisting the positioning of an artificial valve in a blood vessel which is performed by the medical diagnostic imaging apparatus shown in FIG. 1.

FIG. 3 is an explanatory diagram for explaining capturing an image parallel to the extending direction of a blood vessel of interest.

FIG. 4 is an explanatory diagram for explaining simultaneous display of a contrast image and a real-time image.

FIG. 5 is an explanatory diagram for explaining extraction of the sidewalls of a blood vessel and the sidewalls of an artificial valve.

FIG. 6 is an explanatory diagram for explaining extraction of the aspect ratio of the artificial valve.

FIG. 7 is an explanatory diagram for explaining the aspect ratio of the artificial valve in a case where the artificial valve is tilting in the depth direction of the blood vessel.

### DETAILED DESCRIPTION

According to an embodiment, a medical diagnostic imaging apparatus includes: an imaging unit configured to capture a medical image of a blood vessel in which an artificial valve is to be placed; a display unit configured to display the medical image captured by the imaging unit; a storage unit configured to store artificial valve information about a length of the artificial valve; and a judgment unit configured to find a length of the artificial valve in the medical image displayed by the display unit, and to judge whether or not the length thus found and the length in the artificial valve information stored in the storage unit are the same.

According to another embodiment, a medical diagnostic imaging apparatus includes: an imaging unit configured to capture a medical image of a blood vessel in which an artificial valve is to be placed; a display unit configured to display the medical image captured by the imaging unit; a storage unit configured to store artificial valve information about an aspect ratio of the artificial valve; a first judgment unit configured to extract a sidewall of the artificial valve and a sidewall of the blood vessel which are in the medical image displayed by the display unit, and to judge whether or not the sidewall of the artificial valve and the sidewall of the blood vessel are parallel to each other; and a second judgment unit configured to find an aspect ratio of the artificial valve in the medical image displayed by the display unit, and to judge whether or not the aspect ratio thus found and the aspect ratio in the artificial valve information stored in the storage unit are the same.

An embodiment will be described with reference to the drawings.

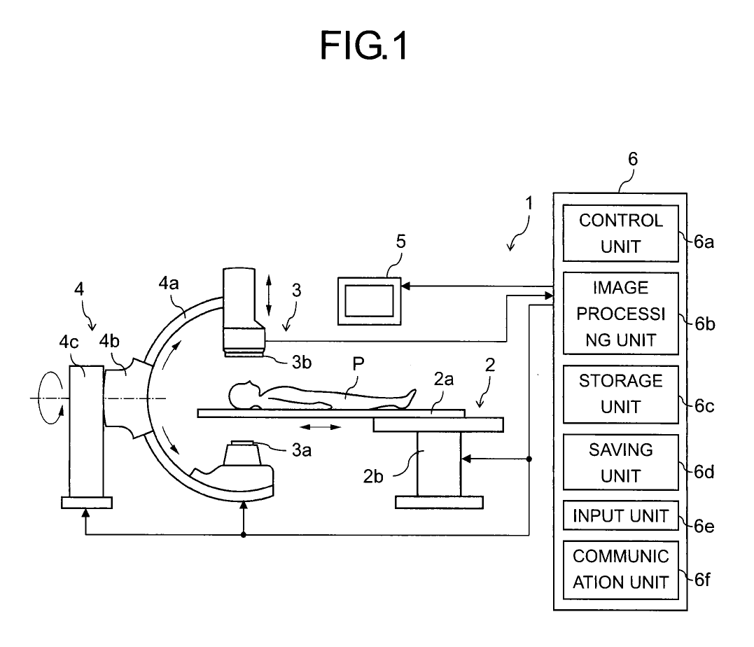

As shown in FIG. 1, a medical diagnostic imaging apparatus 1 according to this embodiment includes: a bed 2 on which to lay a subject P such as a patient; an imaging unit 3 configured to image the subject P on the bed 2; a moving device 4 configured to hold and move the imaging unit 3 to an imaging position; a display unit 5 configured to display an image such as a medical image; and a control device 6 configured to control each part.

The bed 2 includes a rectangular top panel 2a on which to lay the subject P, and a top-panel drive unit 2b configured to support and move the top panel 2a horizontally and vertically. The top-panel drive unit 2b includes a moving mechanism configured to move the top panel 2a, a drive source configured to supply drive power for this movement (both unillustrated), and the like. Such a top-panel drive unit 2b is electrically connected to the control device 6, and its drive is controlled by the control device 6. The bed 2 configured as described moves the subject P on the top panel 2a to a predetermined position by causing the top-panel drive unit 2b to move the top panel 2a to a predetermined height and also move the top panel 2a horizontally.

The imaging unit 3 includes an X-ray irradiation unit 3a configured to irradiate the subject P on the top panel 2a of the bed 2 with X rays, and an X-ray detection unit 3b configured to detect the X rays having passed through the subject P. Being provided movable around the top panel 2a of the bed 2, the imaging unit 3 moves to an imaging position and captures a medical image of a part of interest of the subject P on the top panel 2a from the imaging position. As this medical image, a radiograph of a blood vessel or the like is captured, for example.

The X-ray irradiation unit 3a includes an X-ray tube configured to emit X rays, an X-ray diaphragm configured to focus the X rays emitted from the X-ray tube (both unillustrated), and the like. As the X-ray diaphragm, a collimator or the like is used, for example. Such an X-ray irradiation unit 3a is electrically connected to the control device 6 through a high-voltage generation unit (unillustrated), and its drive is controlled by the control device 6. The X-ray irradiation unit 3a configured as described emits X rays with the X-ray tube, focuses the X rays with the X-ray diaphragm, and irradiates the subject P on the top panel 2a of the bed 2 with the X rays.

Meanwhile, the high-voltage generation unit is a device configured to generate a high voltage to be supplied to the X-ray irradiation unit 3a. The high-voltage generation unit boosts and rectifies a voltage given from the control device 6 and supplies the resultant voltage to the X-ray irradiation unit 3a. Note that for the X-ray irradiation unit 3a to generate desired X rays, the control device 6 controls various conditions on the waveform of the voltage, i.e. amplitude, pulse width, and the like of the voltage to be given to the high-voltage generation unit.

The X-ray detection unit 3b is provided to the moving device 4 in such a way as to face the X-ray irradiation unit 3a and is formed movable toward and away from the facing X-ray irradiation unit 3a. Such an X-ray detection unit 3b is electrically connected to the control device 6 and transmits the X rays it detects, i.e. X-ray image signals to the control device 6. As the X-ray detection unit 3b, an image intensifier, an X-ray flat panel detector (FPD), or the like is used, for example.

The moving device 4 includes: a holding arm 4a configured to hold the X-ray irradiation unit 3a and the X-ray detection 3b in such postures as to make them face each other; an arm support 4b configured to support the holding arm 4a slidably movably; and a support column 4c configured to support the arm support 4b rotatably. Such a moving device 4 is electrically connected to the control device 6, and its drive is controlled by the control device 6.

The holding arm 4a is a C arm in the shape of C, for example, and provided to the arm support 4b slidably movably in the direction the arm extends. At both longitudinal ends of such a holding arm 4a, the X-ray irradiation unit 3a and the X-ray detection unit 3b are provided in the facing postures. Moreover, the arm support 4b is a member to hold the holding arm 4a slidably movably and provided to the support column 4c rotatably. The support column 4c is a member to support the arm support 4b rotatably and provided standing on a floor surface.

The display unit 5 is a display device configured to display various kinds of images such as a medical image of the subject P. In FIG. 1, one display unit 5 is provided as an example. As such a display unit 5, a liquid crystal display, a cathode ray tube (CRT) display, or the like is used, for example.

The control device 6 includes: a control unit 6a such as a microprocessor configured to control each part; an image processing unit 6b configured to create a medical image on the basis of X-ray image signals from the X-ray detection unit 3b; a storage unit 6c configured to store various programs and various data; a saving unit 6d configured to save medical images; an input unit 6e configured to receive input operations from the operator such as a surgeon or an assistant; and a communication unit 6f configured to perform communications with external apparatuses through a communication line such as a network.

The control unit 6a controls each part on the basis of the various programs and the various data stored in the storage unit 6c. Specifically, the control unit 6a controls the bed 2, the imaging unit 3, and the moving device 4 in response to input operations from the operator through the input unit 6e. Moreover, the control unit 6a executes a series of data processing for calculating or processing various data, image display processing for displaying an image such as a medical image, and the like on the basis of various programs. Note that the control unit 6a is capable of obtaining positional information on the holding arm 4a on the basis of an output value from an encoder provided to a drive unit (e.g. servomotor) of the moving device 4 or the like.

The image processing unit 6b creates a medical image out of the X-ray image signals outputted from the X-ray detection unit 3b, and saves the medical image in the saving unit 6d or in a different storage device connected to a network through the communication unit 6f or the like.

The storage unit 6c includes a memory in which to store the various programs to be executed by the control unit 6a and the various data, a memory which functions also as a work area for the control unit 6a, and the like. As such a storage unit 6c, a ROM, a RAM, a magnetic disk device, a semiconductor disk device (flash memory), or the like is used, for example.

The saving unit 6d is a storage device to save medical images sequentially. As such a saving unit 6d, a magnetic disk device, a semiconductor disk device (flash memory), or the like is used, for example. Note that the saving unit 6d may be connected to a communication line such as a network.

The input unit 6e is an operation unit with which the operator performs input operations. As such an input unit 6e, input devices such as a joystick, a keyboard, and a mouse are used, for example. The operator such as a surgeon or an assistant performs input operations through the input unit 6e to move the X-ray irradiation unit 3a and the X-ray detection unit 3b constituting the imaging unit 3 to desired imaging positions.

The communication unit 6f is a device configured to perform communications with external apparatuses through a network such as a local area network (LAN) or the Internet. As such a communication unit 6f, a LAN card, a modem, or the like is used, for example. Moreover, the external apparatuses are an X-ray CT apparatus, a medical image saving apparatus (image server apparatus), and the like.

Next, description will be given of a process of assisting the positioning of an artificial valve in a blood vessel which is performed by the medical diagnostic imaging apparatus 1 described above.

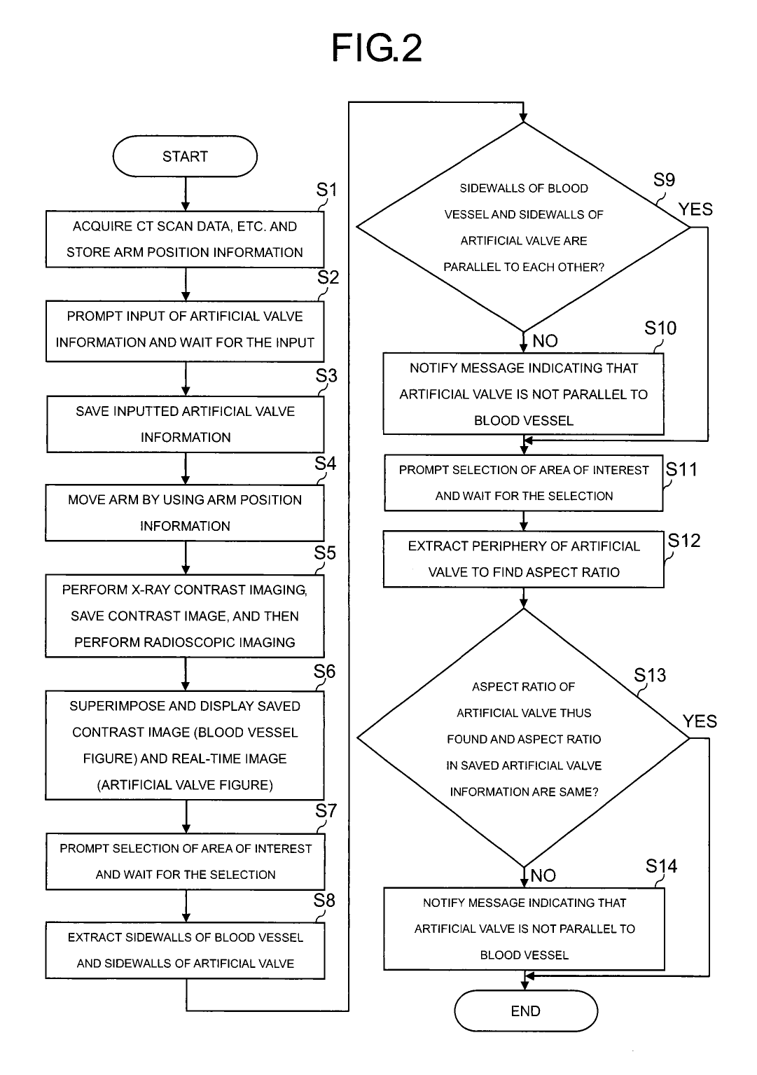

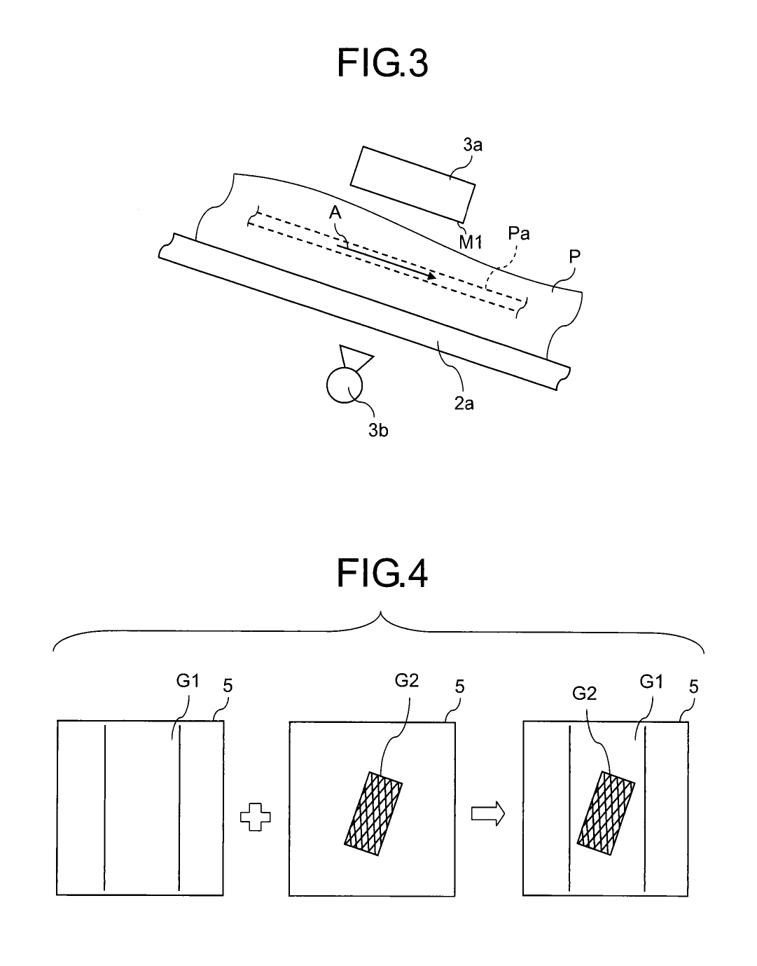

As shown in FIG. 2, the control unit 6a firstly acquires CT scan data and the like from the external apparatuses such as the X-ray CT apparatus through the communication unit 6f, and saves arm position information in the storage unit 6c on the basis of the acquired data (step S1). Obtained from this arm position information is an arm position at which the X-ray irradiation unit 3a is positioned to be capable of irradiating the subject P on the top panel 2a with X rays, and a detection surface M1 (a surface on which X rays fall), or the front surface, of the X-ray detection unit 3b is parallel to the extending direction of a blood vessel Pa as shown in FIG. 3 (see an arrow A in FIG. 3).

Then, through the display unit 5, the control unit 6a notifies the operator of information prompting him or her to input artificial valve information, and waits for the input of the artificial valve information (step S2). The operator inputs, for example, the size of an artificial valve such as the length and width thereof in particular by operating the input unit 6e such a numerical keypad for inputting numbers. Upon input of the artificial valve information about the aspect ratio of the artificial valve, the control unit 6a saves the inputted artificial valve information in the storage unit 6c (step S3).

Thereafter, the control unit 6a uses the arm position information saved in step S1 to cause the moving device 4 to move the holding arm 4a so as to position the imaging unit 3 at an imaging position (step S4). This imaging position is the arm position mentioned earlier at which the detection surface M1 of the X-ray detection unit 3b is parallel to the extending direction of the blood vessel Pa in which the artificial valve is placed (see FIG. 3). Imaging from this position will provide a blood vessel figure parallel to the extending direction of the blood vessel Pa to be subjected to the artificial valve placement (a sectional image parallel to the extending direction of the blood vessel Pa).

Once the holding arm 4a is moved, the control unit 6a causes the imaging unit 3 to perform X-ray contrast imaging, and saves the captured contrast image in the saving unit 6d, and thereafter performs radioscopic imaging (step S5). In this step, in the X-ray contrast imaging, a blood vessel figure is captured through angiography using an imaging agent, and after the X-ray contrast imaging, the imaging unit 3 performs the radioscopic imaging to capture an artificial valve figure as a real-time image.

Along with the radioscopic imaging, the control unit 6a superimposes and display the contrast image (blood vessel figure) saved in step S5 and the real-time image (artificial valve figure) on the display unit 5 (step S6). For example, as shown in FIG. 4, a blood vessel figure G1, which is the saved contrast image, and an artificial valve figure G2, which is the real-time image, are superimposed and displayed on the display unit 5.

Subsequently, the control unit 6a determines whether or not an area of interest is selected from the medical image displayed by the display unit 5, and waits for the selection of the area of interest (step S7). As shown in FIG. 5, the operator selects, for example, an area of interest R1 by viewing the blood vessel figure G1 and the artificial valve figure G2 displayed on the display unit 5 through an operation of the input unit 6e such as the mouse.

Once the area of interest R1 is selected, the control unit 6a extracts the sidewalls of the blood vessel and the sidewalls of the artificial valve (step S8). For example, as shown in FIG. 5, the sidewalls of the blood figure G1 in the area of interest R1 (bold lines A1 and A2 in FIG. 5) and the sidewalls of the artificial valve figure G2 in the area of interest R1 (bold lines B1 and B2 in FIG. 5) are extracted by image processing of the control unit 6a.

Thereafter, the control unit 6a determines whether or not the sidewalls of the blood vessel and the sidewalls of the artificial valve are parallel to each other (step S9). If determining that the sidewalls of the blood vessel and the sidewalls of the artificial valve are not parallel to each other (NO in step S9), the control unit 6a judges that the artificial valve is not parallel to the extending direction of the blood vessel, and notifies the operator such as a surgeon or an assistant of such information (message) through the display unit 5 (step S10). If, on the other hand, determining that the sidewalls of the blood vessel and the sidewalls of the artificial valve are parallel to each other (YES in step S9), the control unit 6a judges that the artificial valve is parallel to the extending direction of the blood vessel, and causes the process to proceed directly to step S11.

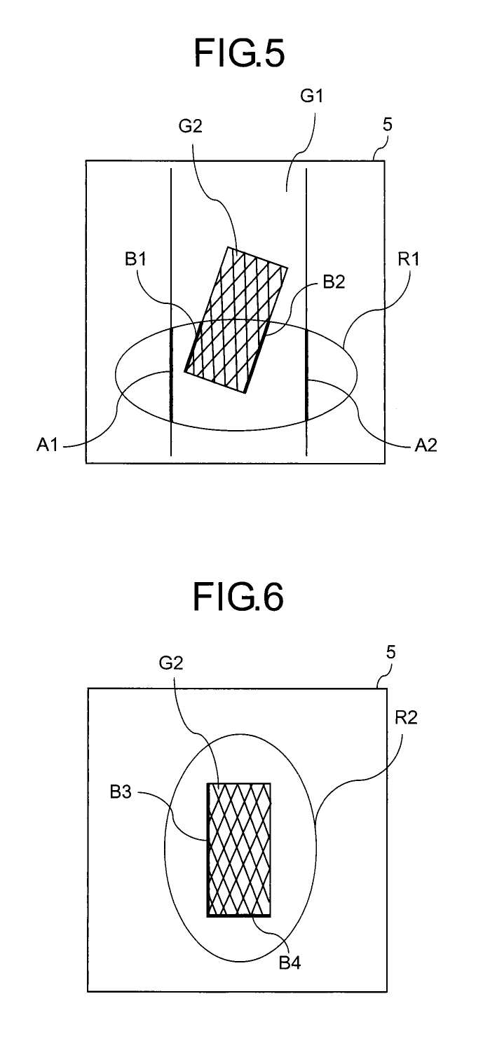

Subsequently, the control unit 6a determines whether or not a next area of interest is selected, and waits for the selection of this area of interest (step S11). As shown in FIG. 6, the operator selects an area of interest R2 by viewing the artificial valve figure G2 displayed on the display unit 5 through an operation of the input unit 6e such as the mouse. Note that while the blood vessel figure G1 is not displayed in FIG. 6, the display is not limited to this case, and the blood vessel G1 may be displayed as well. Nonetheless, the area of interest R2 can be selected easily when the blood vessel figure G1 is not displayed.

Once the area of interest R2 is selected, the control unit 6a extracts the periphery of the artificial valve to find the aspect ratio of the artificial valve (step S12). For example, as shown in FIG. 6, the aspect ratio is found by extracting the periphery of the artificial valve in the area of interest R2 and extracting, based on this periphery, a longitudinal edge (a bold line B3 in FIG. 6) and a widthwise edge (a bold line B4 in FIG. 6) of the artificial valve figure G2 in the area of interest R2 through image processing.

Subsequently, the control unit 6a determines whether or not the aspect ratio of the artificial valve thus found and the aspect ratio in the artificial valve information saved in step S2 are the same (step S13). If determining that the found aspect ratio of the artificial valve and the aspect ratio in the artificial valve information saved in step S2 are different from each other (NO in step S13), the control unit 6a judges that the artificial valve is not parallel to the extending direction of the blood vessel, and notifies the operator such as the surgeon or the assistant of such information (message) through the display unit 5 (step S14). If, on the other hand, determining that the found aspect ratio of the artificial valve and the aspect ratio in the artificial valve information saved in step S2 are the same (YES in step S13), the control unit 6a judges that the artificial valve is parallel to the extending direction of the blood vessel, and ends the process with no further operations.

To be specific, when the found aspect ratio of the artificial valve and the aspect ratio in the artificial valve information saved in step S2 are the same, the artificial valve in the medical image is not tilting either to a near side or a far side and is parallel to the extending direction of the blood vessel. On the other hand, when the found aspect ratio of the artificial valve and the aspect ratio in the artificial valve information saved in step S2 are different from each other, the artificial valve in the medical image is tilting to the near side or the far side in the depth direction of the blood vessel and is not parallel to the extending direction of the blood vessel.

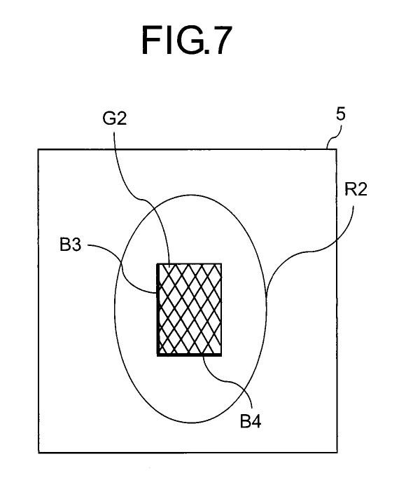

For example, as shown in FIG. 7, the artificial valve figure G2 tilting in the depth direction of the blood vessel is smaller in area than an artificial valve figure parallel to the extending direction of the blood vessel. Here, assuming that the artificial valve figure G2 in FIG. 6 is an artificial valve figure parallel to the extending direction of the blood vessel, the area of the artificial valve figure G2 in FIG. 7 is smaller than the area of the artificial valve figure G2 in FIG. 6, and the aspect ratio of the artificial valve figure G2 in FIG. 7 is different from the aspect ratio of the artificial valve figure G2 in FIG. 6. Thus, the artificial valve figure G2 in FIG. 7 is an artificial valve figure tilting in the depth direction of the blood vessel and not parallel to the extending direction of the blood vessel. Note that when the artificial valve is tilting in the depth direction of the blood vessel, the area of the artificial valve figure G2 displayed is always smaller than the area of the artificial valve figure parallel to the extending direction of the blood vessel.

As described, in the process of assisting the positioning of an artificial valve, firstly, the parallelism between the blood vessel and the artificial valve is checked in the plane direction on a plan view on the display unit 5, and thereafter, the parallelism between the blood vessel and the artificial valve is checked likewise in the depth direction of the blood vessel on the plan view on the display unit 5. Thus, that the artificial valve is not parallel to the extending direction of the blood vessel is notified when the artificial valve in the medical image is tilting in the plane direction and also when the artificial valve in the medical image is tilting in the depth direction of the blood vessel. It is therefore possible to check the parallelism between the blood vessel and the artificial valve in the depth direction of the blood vessel by using the plan view on the display unit 5. Accordingly, it is possible to assist the placement of the artificial valve at an accurate position inside the blood vessel.

Here, the control unit 6a functions as a first judgment unit configured to judge whether or not the sidewalls of the artificial valve and the sidewalls of the blood vessel are parallel to each other, and as a second judgment unit configured to judge whether or not the found aspect ratio of the artificial valve and the aspect ratio in the artificial valve information in the storage unit 6c are the same. Moreover, the display unit 5 functions as a notification unit configured to notify that the artificial valve is not parallel to the extending direction of the blood vessel.

Meanwhile, that the artificial valve is not parallel to the extending direction of the blood vessel may be notified in a different display fashion than the message mentioned earlier. The artificial valve may be displayed by changing its color in accordance with to which one of the near side and the far side the artificial valve is tilting. Alternatively, words such as “near side” or “far side” may be displayed, or a relationship diagram showing the positional relationship between the blood vessel and the artificial valve may be displayed. In these cases, it is possible to visually recognize that the artificial valve is tilting in the depth direction, and also whether the artificial valve is tilting to the near side or to the far side. However, that the artificial valve is not parallel to the blood vessel (the artificial valve and the blood vessel are off parallel) will not be displayed when a tolerance given to the posture of the artificial valve, for instance, when the amount of misalignment between the blood vessel and the artificial valve in the front, rear, right, and left directions is ±3%, for example.

Here, in one example of displaying the artificial valve by changing its color, a part of the artificial valve which is misaligned is displayed in a different color from the remaining part. Further, in this display, the amount of misalignment may also be displayed in terms of percentage (%) (including the display of front, rear, right, and left and the plus/minus sign). In this case, the amount of the misalignment is displayed as “Front-Rear: Front +150”, Left-Right: Right +80,” for example. Moreover, in one example of displaying “near side” or “far side,” a sentence such as “misaligned to near side” or “misaligned to far side” is displayed. Further, in the case of displaying the relationship diagram showing the positional relationship between the blood vessel and the artificial valve, a relationship diagram showing a positional relationship reflecting a side view of the displayed artificial valve is displayed.

As described above, according to this embodiment, the control unit 6a finds the aspect ratio of the artificial valve from the displayed medical image, and determines whether or not this aspect ratio and the aspect ratio in the artificial valve information stored in the storage unit 6c are the same. Then, if determining that the aspect ratio thus found and the aspect ratio in the artificial valve information are the same, the control unit 6a judges that the artificial valve is parallel to the extending direction of the blood vessel. On the other hand, if determining that the found aspect ratio and the aspect ratio in the artificial valve information are different from each other, the control unit 6a judges that the artificial valve is not parallel to the extending direction of the blood vessel, and notifies such information through the display unit 5.

Thus, that the artificial valve is not parallel to the extending direction of the blood vessel is notified also when the artificial valve in the medical image is tilting in the depth direction. Accordingly, it is possible to check the parallelism between the blood vessel and the artificial valve in the depth direction of the blood vessel with the plan view on the display unit 5. Specifically, the imaging does not need to be performed multiple times by changing the imaging position along the circumference of the blood vessel. Thus, the time and effort in checking the position can be reduced. Accordingly, it is possible to assist the placement of the artificial valve at an accurate position inside the blood vessel without taking much time and effort.

Meanwhile, the medical image mentioned above may be enlarged or reduced in some cases when necessary. However, even in such cases, the judgment can be made accurately without being affected by the enlargement or reduction of the medical image since the aspect ratio of the artificial valve is used to judge the parallelism of the artificial vale in the depth direction of the blood vessel.

In the foregoing embodiment, the control unit 6a judges whether or not the aspect ratio of the artificial valve and the aspect ratio in the artificial valve information in the storage unit 6c are the same, as one way of judging whether or not the artificial valve is parallel to the extending direction of the blood vessel. Note, however, that the judgment is not limited to the above way. For example, the control unit 6a may save artificial valve information about the length of the artificial valve in the storage unit 6c, find the length of the artificial valve in the medical image displayed by the display unit 5, and judge whether or not the length thus found and the length in the artificial valve information in the storage unit 6c are the same. In this case, the artificial valve is found to be not parallel to the extending direction of the blood vessel when it is judged that the found length and the length in the artificial valve information in the storage unit 6c are not the same. Accordingly, the same advantageous effect as that mentioned above can be obtained. In this case, the control unit 6a functions as a judgment unit configured to judge whether or not the found length and the length in the artificial valve information stored in the storage unit 6c are the same.

Moreover, in the foregoing embodiment, the control unit 6a performs the process including: determining whether or not the sidewalls of the artificial valve and the sidewalls of the blood vessel are parallel to each other; finding the aspect ratio of the artificial valve if the sidewalls of the artificial valve and the sidewalls of the blood vessel are determined as being parallel to each other; and determining whether or not the aspect ratio thus found and the aspect ratio in the artificial valve information stored in the storage unit 6c are the same. However, the process is not limited to the above way. The control unit 6a may simultaneously perform a process of determining whether or not the sidewalls of the artificial valve and the sidewalls of the blood vessel are parallel to each other, and a process of finding the aspect ratio of the artificial valve and determining whether or not the aspect ratio thus found and the aspect ratio in the artificial valve information stored in the storage unit 6c are the same. In this case, in FIG. 5, for example, an area of interest is selected in such a way as to include the sidewalls of the blood vessel and the periphery of the artificial valve. By such parallel processing, the processing time can be shortened.

Moreover, in the foregoing embodiment, the control unit 6a notifies that the artificial valve is not parallel to the extending direction of the blood vessel by displaying such information on the display unit 5. However, the notification is not limited to the above away. For example, the control unit 6a may make the notification by means of a sound such as buzzer sound or voice or of light of a lamp or the like. In this case, a device which notifies that the artificial valve is not parallel to the extending direction of the blood vessel by means of sound or light functions as the notification unit. Note that using the display unit 5 as the notification unit as in the embodiment described above eliminates the necessity for adding another device as the notification unit, and therefore makes it possible to achieve low cost.

Moreover, in the foregoing embodiment, the control unit 6a notifies only that the artificial valve is not parallel to the extending direction of the blood vessel. However, the information to be notified is not limited to the above case. For example, misalignment information such as the amount of tilt of the artificial valve may be notified. In this case, the surgeon or the like can correct the position of the artificial valve on the basis of such misalignment information. Accordingly, it is possible to provide more secure assistance for the placement of the artificial valve at an accurate position inside the blood vessel.

Moreover, in the foregoing embodiment, the control unit 6a uses the aspect ratio of the artificial valve to judge whether or not the artificial valve is parallel to the extending direction of the blood vessel, that is, whether or not the artificial valve is tilting in the depth direction of the blood vessel. However, the judgment is not limited to the above way. For example, the control unit 6a may use the area or diagonal line of the artificial valve to judge whether or not the artificial valve is tilting in the depth direction of the blood vessel. Note that in this case, since the size of the artificial valve varies along with the enlargement or reduction of the medical image, the area or diagonal line of the artificial valve needs to be used after enlarging or reducing it in accordance with the enlargement or reduction ratio of the medical image.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical diagnostic imaging apparatus comprising:
an imaging unit configured to capture a medical image of a blood vessel in which an artificial valve is to be placed;
a display unit configured to display the medical image captured by the imaging unit;
a storage unit configured to store artificial valve information about a length of the artificial valve; and
a judgment unit configured to find a length of the artificial valve in the medical image displayed by the display unit, and to judge whether or not the length thus found and the length in the artificial valve information stored in the storage unit are the same.

2. A medical diagnostic imaging apparatus comprising:
an imaging unit configured to capture a medical image of a blood vessel in which an artificial valve is to be placed;
a display unit configured to display the medical image captured by the imaging unit;
a storage unit configured to store artificial valve information about an aspect ratio of the artificial valve;
a first judgment unit configured to extract a sidewall of the artificial valve and a sidewall of the blood vessel which are in the medical image displayed by the display unit, and to judge whether or not the sidewall of the artificial valve and the sidewall of the blood vessel are parallel to each other; and
a second judgment unit configured to find an aspect ratio of the artificial valve in the medical image displayed by the display unit, and to judge whether or not the aspect ratio thus found and the aspect ratio in the artificial valve information stored in the storage unit are the same.

3. The medical diagnostic imaging apparatus according to claim 2, wherein when the first judgment unit judges that the sidewall of the artificial valve and the sidewall of the blood vessel are parallel to each other, the second judgment unit finds the aspect ratio of the artificial valve in the medical image displayed by the display unit, and judges whether or not the aspect ratio thus found and the aspect ratio in the artificial valve information stored in the storage unit are the same.

4. The medical diagnostic imaging apparatus according to claim 2, further comprising a notification unit configured to notify that the artificial valve is not parallel to an extending direction of the blood vessel, when the first judgment unit judges that the sidewall of the artificial valve and the sidewall of the blood vessel are not parallel to each other.

5. The medical diagnostic imaging apparatus according to claim 3, further comprising a notification unit configured to notify that the artificial valve is not parallel to an extending direction of the blood vessel, when the first judgment unit judges that the sidewall of the artificial valve and the sidewall of the blood vessel are not parallel to each other.

6. The medical diagnostic imaging apparatus according to claim 2, further comprising a notification unit configured to notify that the artificial valve is not parallel to an extending direction of the blood vessel, when the second judgment unit judges that the found aspect ratio and the aspect ratio in the artificial valve information stored in the storage unit are not the same.

7. The medical diagnostic imaging apparatus according to claim 3, further comprising a notification unit configured to notify that the artificial valve is not parallel to an extending direction of the blood vessel, when the second judgment unit judges that the found aspect ratio and the aspect ratio in the artificial valve information stored in the storage unit are not the same.

8. The medical diagnostic imaging apparatus according to claim 1, wherein
the imaging unit includes an X-ray detection unit having a detection surface on which X rays fall, and
the medical diagnostic imaging apparatus further comprises a moving device configured to move the X-ray detection unit to a position at which the detection surface is parallel to the extending direction of the blood vessel.

9. The medical diagnostic imaging apparatus according to claim 2, wherein
the imaging unit includes an X-ray detection unit having a detection surface on which X rays fall, and
the medical diagnostic imaging apparatus further comprises a moving device configured to move the X-ray detection unit to a position at which the detection surface is parallel to the extending direction of the blood vessel.

10. The medical diagnostic imaging apparatus according to claim 3, wherein
the imaging unit includes an X-ray detection unit having a detection surface on which X rays fall, and
the medical diagnostic imaging apparatus further comprises a moving device configured to move the X-ray detection unit to a position at which the detection surface is parallel to the extending direction of the blood vessel.
